(19) United States
(12) Patent Application Publication
(10) Pub. No.: US 2017/0112420 A1
(43) Pub. Date: Apr. 27, 2017

(54) BIO-SENSING APPARATUS AND SENSING METHOD THEREOF

(71) Applicant: GINGY TECHNOLOGY INC., HSINCHU CITY (TW)

(72) Inventors: CHUN-LANG HUNG, HSINCHU CITY (TW); TSUNG-SHAN CHEN, HSINCHU CITY (TW)

(21) Appl. No.: 15/398,327

(22) Filed: Jan. 4, 2017

Related U.S. Application Data
(63) Continuation-in-part of application No. 14/835,130, filed on Aug. 25, 2015.
Application No. 14/835,130 is a continuation-in-part of application No. 15/208,619, filed on Jul. 13, 2016.
Application No. 15/208,619 is a continuation-in-part of application No. 14/978,237, filed on Dec. 22, 2015.

(30) Foreign Application Priority Data
Aug. 26, 2014 (TW) 103129359
Dec. 22, 2014 (TW) 103144744
Sep. 7, 2016 (TW) 105128942

(51) Int. Cl. A61B 5/145; A61B 5/00; A61B 5/1172; A61B 5/1455

(52) U.S. Cl. CPC A61B 5/14532; A61B 5/1455; A61B 5/6826; A61B 5/1172; G06K 9/00013

(57) ABSTRACT

This invention discloses a bio-sensing apparatus and a sensing method thereof. The bio-sensing apparatus comprises at least one light sensor, reflection light module, transmission light module and control module, wherein the bio-sensing apparatus can provide two different lights from the up and down side of the finger, and calculate the blood glucose information by sensing the spectrum to provide the more precision measurement of the blood glucose concentration.

### CROSS-REFERENCE TO RELATED APPLICATION

This application is a Continuation of U.S. patent application Ser. No. 14/835,130 filed on Aug. 25, 2015, Ser. No. 14/978,237 filed on Dec. 22, 2015, and Ser. No. 15/208,619 filed on Jul. 13, 2016. This patent application identified above is incorporated here by reference in its entirety to provide continuity of disclosure, and this application also claims the priority to Taiwan Patent Application No. 105128942 filed in the Taiwan Patent Office on Sep. 7, 2016, and the entirety of each of the above-mentioned patent applications is hereby incorporated by reference herein and made a part of this specification.

### BACKGROUND

1. Technical Field

The present disclosure relates to a bio-sensing apparatus, in particular, to a bio-sensing apparatus and sensing method thereof adapted to sense biometric signals such as blood glucose information.

2. Description of Related Art

Diabetes has become a lifestyle-related disease, and there are more and more people suffering from the illness. To the diabetics, monitoring and controlling the blood glucose level in daily life is absolutely essential, and blood glucose meters are most frequently employed to measure patient's blood glucose level.

Generally, blood glucose meters are categorized into two types which are non-invasive blood glucose meters and invasive blood glucose meters. Whenever the invasive blood glucose meter to measure blood glucose level is used, detecting strips to sample blood have to be used. However, it is not only an uneconomical burden, but also a psychological stress to patients after a long-term usage.

The non-invasive blood glucose meters are restricted to higher technical requirements even though they are user-friendly and can lower the risk of infection. For example, the invasive blood glucose meters employ infrared spectrum to detect blood glucose level, but such technique lacks better measurement precision and stability and cannot be applied effectively.

In addition, employing the infrared spectrum to detect blood glucose level depends on light generated by a light spectrometer and sensors, causing the invasive blood glucose meter to be large in size and expensive in cost. The conventional invasive blood glucose meters therefore fail to meet the market demands and are not economically available.

### SUMMARY

The primary purpose of the present disclosure is to provide a bio-sensing apparatus and sensing method thereof using the non-invasive bio-sensing technique. The present disclosure employs light sensors disposed corresponding to each other to provide lights on and under an object to be sensed to resolve the technical problem of conventional optical sensing techniques incapable of measuring biometric signals accurately by using a single light. By sequentially or simultaneously providing lights, the present disclosure is able to provide more infrared absorption spectrum information, thereby measuring blood glucose level more precisely.

According to one exemplary embodiment of the present disclosure, a bio-sensing apparatus adapted to sense blood glucose information by using one finger is provided, comprising: at least one light sensor sensing a light intensity signal to produce a sensing signal; a reflection light module disposed at the same side as the light sensor for producing a first test light; a transmission light module disposed on the light sensor for generating a second test light, and an accommodating space disposed between the light sensor and the transmission light module; and a control module electrically connected to the light sensor, the reflection light module and the transmission light module, and receiving the sensing signal to detect the blood glucose information.

According to an exemplary embodiment of the invention, the reflection light module and the transmission light module both emit light toward the accommodating space.

According to an exemplary embodiment of the invention, the reflection light module comprises at least one first light array having a plurality of light sources having their respective spectra for generating the first test light, and the first test light has a wavelength range ranging from 700 nm-3000 nm.

According to an exemplary embodiment of the invention, the transmission light module comprises at least one second light array having a plurality of light sources having their respective spectra for generating the second test light, and the second test light has a wavelength range ranging from 700 nm-3000 nm.

According to an exemplary embodiment of the invention, the reflection light module comprises a plurality of first light sources and a plurality of first filters, the plurality of first filters are respectively disposed on the plurality of first light sources for producing a spectrum having different wavebands; and the transmission light module comprises a plurality of second light sources and a plurality of second filters, the plurality of second filters are respectively disposed on the plurality of second light sources for producing a spectrum having different wavebands.

According to an exemplary embodiment of the invention, in a first sensing mode, the reflection light module generates light to the finger in the accommodating space during a first time period, the light sensor senses the received light to produce a first sensing signal, and the transmission light module generates light to the finger in the accommodating space during a second time period, the light sensor senses the received light to produce a second sensing signal, and the bio-sensing apparatus detects the blood glucose information according to the first sensing signal and the second sensing signal, wherein the first time period and the second time period do not overlap.

According to an exemplary embodiment of the invention, in a second sensing mode, the reflection light module and the transmission light module both generate light to the finger in the accommodating space, the light sensor senses the received light to produce a third sensing signal, and the bio-sensing apparatus detects the blood glucose information according to the third sensing signal.

According to an exemplary embodiment of the invention, when the bio-sensing apparatus is in the process of sensing, the reflection light module and the transmission light module sequentially or simultaneously generate light having a plurality of spectra to the finger in the accommodating space, the light sensor senses the received light to produce the sensing signals, and the bio-sensing apparatus produces a reflection spectrum and a transmission spectrum according to the sensing signals and then detects the blood glucose information according to the reflection spectrum and the transmission spectrum.

According to an exemplary embodiment of the invention, a calculation method used to calculate the blood glucose information comprises analysis of variance, regression analysis, or curve fitting.

According to an exemplary embodiment of the invention, the bio-sensing apparatus comprises the control module including: a light controller electrically connected to the reflection light module and the transmission light module for adjusting the spectrum of the first test light and the second test light; an analog/digital converter electrically connected to the light sensor for receiving sensing signals outputted by the light sensor and then transforming the received sensing signals into digital signals, and a processing unit electrically connected to the analog/digital converter for receiving the digital signals to detect the blood glucose information.

According to an exemplary embodiment of the invention, light emitted by the reflection light module and the transmission light module comprises near-infrared and short wavelength infrared.

According to an exemplary embodiment of the invention, the light sensor comprises a semiconductor photosensitive array having a plurality of semiconductor photosensitive units, and each of the semiconductor photosensitive units comprises a first-type doped semiconductor layer and a second-type semiconductor layer.

According to the other exemplary embodiment of the present disclosure, a biometric signals sensing method adapted to sense blood glucose information by using one finger is provided, including the following steps: providing at least one light sensor; providing at least one reflection light module, wherein the reflection light module and the light sensor are disposed at the same side; providing a transmission light module disposed on the light sensor; an accommodating space provided between transmission light module and the light sensor for accommodating the finger; sequentially or simultaneously turning on the reflection light module and the transmission light module to generate a plurality of test lights having their respective spectra to the finger in the accommodating space; sensing light transmitting through and reflected off of the finger to producing a sensing signal; and producing a reflection spectrum and a transmission spectrum according to the sensing signal, and detecting blood glucose information according to the reflection spectrum and the transmission spectrum.

To sum up, the bio-sensing apparatus and sensing method thereof of the present disclosure sequentially or simultaneously provides light having different spectra on and under a finger to be sensed to respectively obtain the spectrum with respect to light transmitting through and reflected off of the finger or to provide the spectrum information obtained from the compound light, thereby detecting blood glucose information more accurately. As the bio-sensing apparatus of the present disclosure may be made of semiconductor optoelectronic components to have a small size and a light weight, it can be integrated in handheld devices such as cell phones. In addition, the present disclosure employs the non-invasive technique to detect blood glucose level so as to resolve the technical problem of the conventional non-invasive blood glucose meters that are costly and lack measurement precision.

In order to further understand the techniques, means and effects of the present disclosure, the following detailed descriptions and appended drawings are hereby referred to, such that, and through which, the purposes, features and aspects of the present disclosure can be thoroughly and concretely appreciated; however, the appended drawings are merely provided for reference and illustration, without any intention to be used for limiting the present disclosure.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 is a schematic diagram illustrating the structure of one embodiment of the bio-sensing apparatus of the present disclosure.

FIG. 2 is a function block diagram of one embodiment of the bio-sensing apparatus of the present disclosure.

FIG. 3A is a schematic diagram of one embodiment of the light array of the present disclosure.

FIG. 3B is a schematic diagram of the other embodiment of the light array of the present disclosure.

FIG. 4 is a flow chart of one embodiment of the biometric signals sensing method of the present disclosure.

FIG. 5 is a flow chart of another embodiment of the biometric signals sensing method of the present disclosure.

FIG. 6 is a flow chart of yet another embodiment of the biometric signals sensing method of the present disclosure.

FIG. 7 is a flow chart of an embodiment of the biometric signals sensing method of the present disclosure.

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

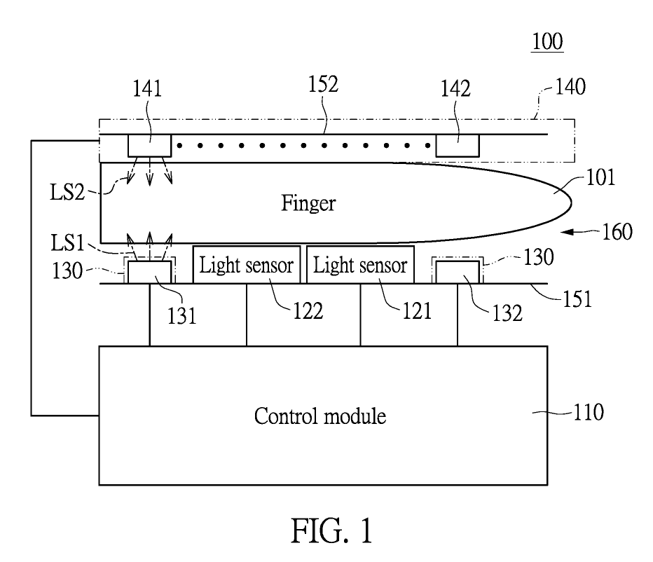

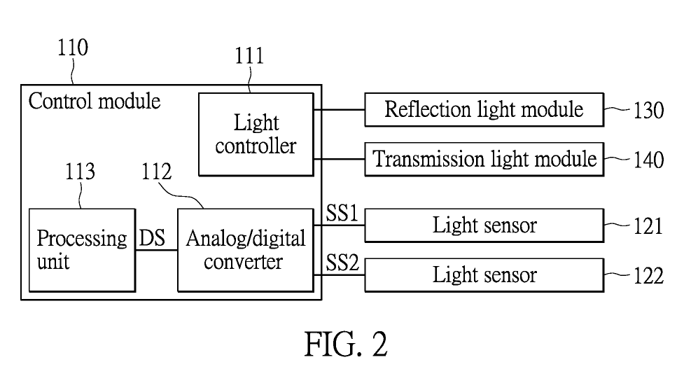

Please refer to FIG. 1 and FIG. 2 which are respectively a schematic diagram illustrating the structure of one embodiment of the bio-sensing apparatus of the present disclosure and a function block diagram of one embodiment of the bio-sensing apparatus of the present disclosure. A bio-sensing apparatus 100 includes a control module 110, light sensors 121, 122, a reflection light module 130, and a transmission light module 140. The control module is electrically connected to the light sensors 121, 122, the reflection light module 130 and the transmission light module 140 for controlling the spectra produced by the reflection light module 130 and the transmission light module 140 and receiving sensing signals SS1, SS2 sensed by the light sensors 121, 122 so as to calculate a sensing spectrum such as the infrared absorption spectrum according to the received sensing signals SS1, SS2. After that, the control module 110 detects blood glucose information according to the spectrum information and then outputs the blood glucose information or the spectrum information according to the actual requirements.

In an exemplary embodiment, the reflection light module 130 and the transmission light module 140 respectively generate a first test light LS1 under (finger pulp) and a second test light LS2 on (back of the finger) a finger 101, wherein the first test light LS1 and the second test light LS2 have their respective spectra which is adjusted in a specific waveband. The bio-sensing apparatus 100 uses the spectrum information with respect to light transmitting through or reflected off of the finger 101 sensed by the light sensors 121, 122 to calculate biometric signals.

The light sensors 121, 122 and the transmission light module 140 are disposed at the same side for generating the first test light LS1 reflected from the finger 101. The transmission light module 140 is disposed on the light sensors 121, 122 for generating the second test light LS2 transmitting through the finger 101. An accommodating space 160 is provided between the transmission light module 140 and the light sensors 121, 122 for accommodating the finger 101. The reflection light module 130 and the transmission light module 140 both emit light toward the accommodating space 160 in which the finger 101 is accommodated. In other words, lights are respectively provided on and under the finger 101 for providing the reflection light and the transmission light which are used to detect the blood glucose information. The light sensors 121, 122 and the reflection light module 130 may be disposed on a lower circuit board 151 and the transmission light module 140 may be disposed on an upper circuit board 152, and the present embodiment does not limit the disposition thereof.

The lights respectively emitted by the reflection light module 130 and the transmission light module 140 can emit to the finger 101 through a press sheet or an optical dielectric layer (not shown). For example, the lights may be emitted to the finger 101 through a light guide element, but the present disclosure is not limited thereto.

In the present embodiment, the bio-sensing apparatus 100 includes one or more light sensors 121, 122, and each of the light sensors 121, 122 is used to sense light intensity signals having different wavebands (wavelength ranges) to produce the sensing signals SS1, SS2 to the control module 110. In addition, the light sensors 121, 122 can respectively sense lights having different wavelength ranges. In the present embodiment, different light sensors are combined to sense the desired waveband. For example, the light sensors 121, 122 can sense a waveband of electromagnetic radiation ranging from 700 nn-3000 nm which includes the spectral range of near infrared (NIR) and short wavelength infrared. In different embodiments, the light sensors 121, 122 can sense wavebands defined according to the desired wavelength range, but the present disclosure is not limited thereto. The light sensors 121, 122 are controlled to sense light intensity signals respectively or simultaneously.

The light sensors 121, 122 may be a photoelectric sensor or an infrared receiver used to transform light intensity signals into electrical signals, but the present disclosure is not limited thereto. For example, the light sensors 121, 122 may be a sensing array formed of a plurality of semiconductor elements, wherein structure of each of the plurality of semiconductor elements is a PN interface made of two layers of doped semiconductor. The doped semiconductor may be semiconductor material doped with N-type dopant and semiconductor material doped with P-type dopant, wherein the semiconductor material may be made of silicon including one of single crystal silicon, polycrystal silicon, amorphous silicon, or microcrystal silicon. The semiconductor material may also be CuInGaSe₂ (CIGS), CdS, CdSe, GaAs, InGaAs, InP, CuInSe2 (CIS), CdTe, InP, and semiconductor organic material, or a multi-layered structure stacked by a combination thereof. Material of the light sensors 121, 122 is not limited thereto. The N-type dopant doped in the silicon semiconductor material may be an element of group V listed in the Periodic Table, and the P-type dopant doped in the semiconductor material may be an element of group III listed in the Periodic Table, wherein the element of group V includes P, As, Sb, and so on, and the element of group III includes B, Al, Ga, In, and so on.

The light sensors 121, 122 made of a single semiconductor element can sense light intensity signals having a specific waveband. The sensing array made of semiconductor elements having their respective wavebands can sense a larger waveband range, and the sensing array made of semiconductor elements having the same waveband can also be applied to the fingerprint identification apparatus. In other words, the light sensors 121, 122 of the present disclosure can be applied to an optical fingerprint identification apparatus, but the present disclosure is not limited thereto.

The reflection light module 130 and the transmission light module 140 can output test light having different wavebands, wherein the wavelength range is between 700 nm and 3000 nm which includes the wavelength range of near infrared and short wavelength infrared. The reflection light module 130 may be made of one or more light arrays 131, 132 having different wavelengths for producing the desired spectrum, and the transmission light module 140 may be made of one or more light arrays 141, 142 having different wavelengths for producing the desired spectrum.

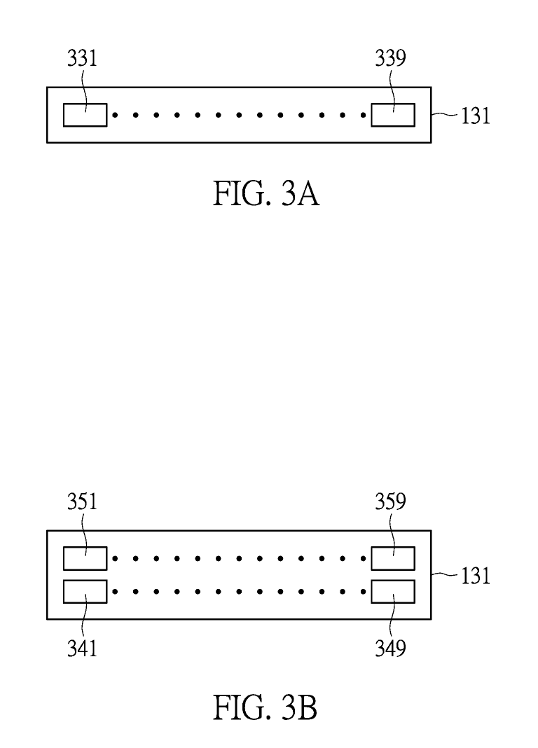

The light arrays 131, 132, 141, 142 can be accomplished by different methods. Please refer to FIG. 3A which is a schematic diagram of one embodiment of the light array of the present disclosure. The light array 131 is used as an example for describing details. The light array 131 may be made of a plurality of solid-state light emitting elements 331-339 (e.g. light emitting diodes (LED)) having their respective spectra. By selecting and combining different spectra, the light array 131 can have a spectral range ranging from 700 nm-3000 nm for meeting the actual requirements. Please refer to FIG. 3B which is a schematic diagram of another embodiment of the light array of the present disclosure. The light array 131 is used as an example for describing details. The light array 131 is formed of a plurality of light sources 341-349 having all waveband spectra and a plurality of filters 351-359 covering the plurality of light sources 341-349. The plurality of light sources 341-349 having all waveband spectra have a wider spectral region such as 700 nm-3000 nm, and the plurality of filters 351-359 can select wavelength and filter lights without having a specific wavelength. For example, if the filter 351 has a waveband of 700 nm-1000 nm, lights emitted by the light source 341 cannot transmit through the filter 351, except for the light having the wavelength of 700 nm-1000 nm. If the filter 359 has a waveband of 2700 nm-3000 nm, when lights emitted by the light source 349 enter the filter 359, only the light having the wavelength of 2700 nm-3000 nm can transmit therethrough. In other words, by selecting the plurality of filters 351-359 having their respective wavebands, the light array 131 can produce a spectrum having different wavebands, thereby generating test light having the desired waveband range. Structures of the light arrays 132, 141, 142 are the same as that of the light array 131, and unnecessary details are not repeated.

The first test light LS1 outputted by the reflection light module 130 has a waveband ranging from 700 nm-3000 nm, a spectrum thereof can be selected from a range such as 700 nm-900 nm, but the present disclosure is not limited thereto. Similarly, the second test light LS2 outputted by the reflection light module 140 has a waveband ranging from 700 nm-3000 nm, a spectrum thereof can be selected from a range such as 700 nm-900 nm, but the present disclosure is not limited thereto. In the reflection light module 130 and the transmission light module 140, the light sources 331-339 and 341-349 are respectively controlled to adjust the spectrum respectively outputted by the reflection light module 130 and the transmission light module 140, thereby producing test lights having different spectra to the finger 101 to detect the blood glucose information.

The control module 110 includes a light controller 111, an analog/digital converter 112 and a processing unit 113. The light controller 111 is electrically connected to the reflection light module 130 and the transmission light module 140 for controlling the spectrum and the time-period outputted therefrom. The analog/digital converter 112 is electrically connected to the processing unit 113 and the light sensors 121, 122 for transforming the sensing signals SS1, SS2 outputted by the light sensors 121, 122 into digital signals DS. The processing unit 113 receives and then calculates the digital signals DS to obtain the blood glucose information. The calculation method includes infrared absorption spectrum, analysis of variance, regression analysis, curve fitting, and so on, but the present disclosure is not limited thereto.

The control module 110 and the light sensors 121, 122 can be disposed on the same circuit boards 151, 152 or integrated in the same chip. In addition, the control module 110 can be installed in cell phones, computers or tablet computers, and so on, but the present disclosure is not limited thereto.

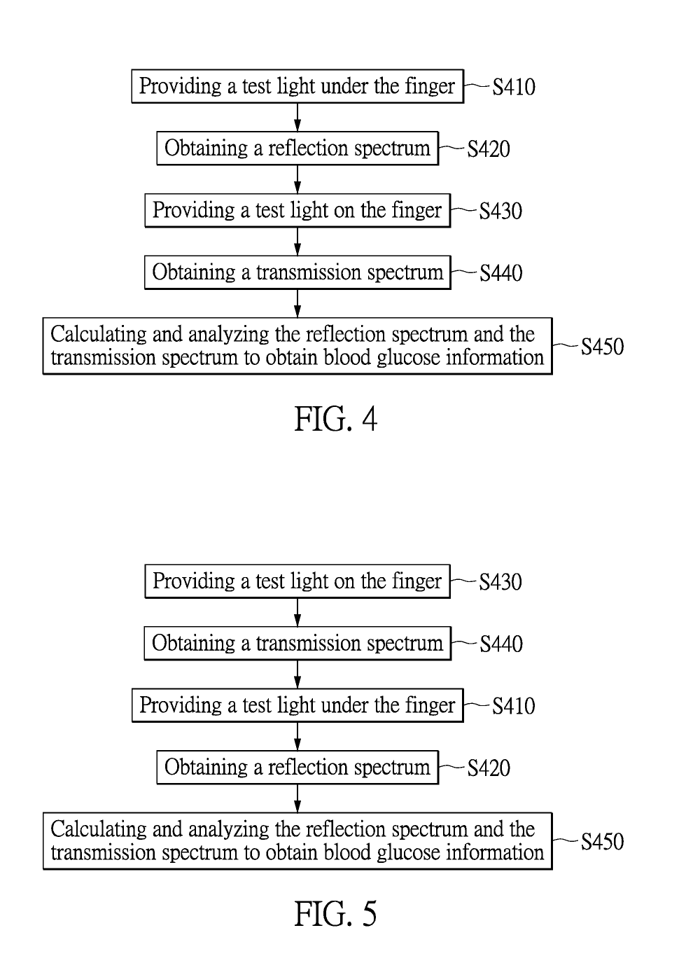

The bio-sensing apparatus 100 may have different sensing modes. In a first sensing mode, the test lights are respectively provided under and on the finger 101 to obtain the reflection spectrum and transmission spectrum, and then the spectra are calculated to obtain the blood glucose information. Please refer to FIG. 4 which is a flow chart of one embodiment of the biometric signals sensing method of the present disclosure. The steps are as follows. In S410: turning on the reflection light module 130 to provide the first test light LS1 under the finger 101; in S420: the light sensors 121, 122 sensing the light reflected off of the finger 101 to output the sensing signals SS1, SS2 to the control module 110, and the control module 110 obtaining a reflection spectrum such as infrared absorption spectrum according to the sensing signals SS1, SS2; in S430: turning on the transmission light module 140 to provide the second test light LS2 on the finger 101; in S440: the control module 110 obtaining a transmission spectrum according to the sensing signals SS1, SS2; and in S450: the bio-sensing apparatus 100 calculating and analyzing the reflection spectrum and the transmission spectrum to obtain blood glucose information.

In S410-S440, the spectral range of the first test light LS1 and the second test light LS2 are defined according to the actual requirements. For example, the spectral range can be scanned from a short wavelength to a high wavelength or from a high wavelength to a short wavelength, or electromagnetic radiation having a specific wavelength can be selectively outputted, thereby obtaining variations of the spectrum in the desired spectral range.

Please refer to FIG. 5 which is a flow chart of another embodiment of the biometric signals sensing method of the present disclosure. The difference between FIG. 5 and FIG. 4 is that the test light is provided on the finger 101 and then under the finger 101. The sequence of S410 and S420 and that of S430 and S440 are exchanged. And in S450, the bio-sensing apparatus 100 calculates and analyzes the reflection spectrum and the transmission spectrum to obtain the blood glucose information. For the other steps of FIG. 5 refer to FIG. 4, and unnecessary details are not repeated.

As shown in FIG. 4 and FIG. 5, the reflection light module 130 and the transmission light module 140 are turned on at different time periods to respectively provide the test light to the finger 101, wherein the time period of the reflection light module 130 emitting light and that of the transmission light module 140 do not overlap.

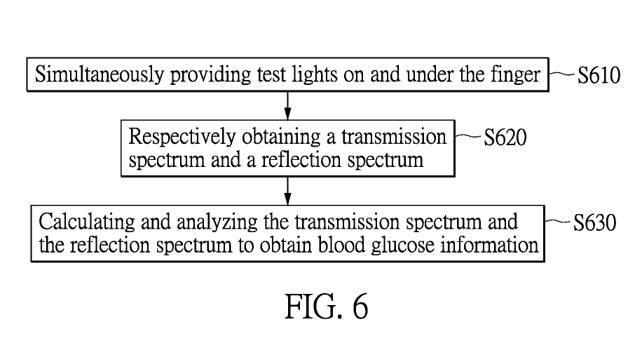

In a second sensing mode, the bio-sensing apparatus 100 provides the test lights on and under the finger 101 simultaneously. Please refer to FIG. 6, which is a flow chart of yet another embodiment of the biometric signals sensing method of the present disclosure. In S610: the reflection light module 130 and the transmission light module 140 simultaneously provide the first test light LS1 and the second test light LS2 to the finger 101; in S620: the light sensors 121, 122 sensing the light received by the finger 101 and then outputting the sensing signals S1, SS2 to the control module 110; and in S630: the control module 110 obtaining a reflection spectrum and a transmission spectrum according to the sensing signals SS1, SS2. After that, in S630, the bio-sensing apparatus 100 calculates and analyzes the reflection spectrum and the transmission spectrum to obtain the blood glucose information.

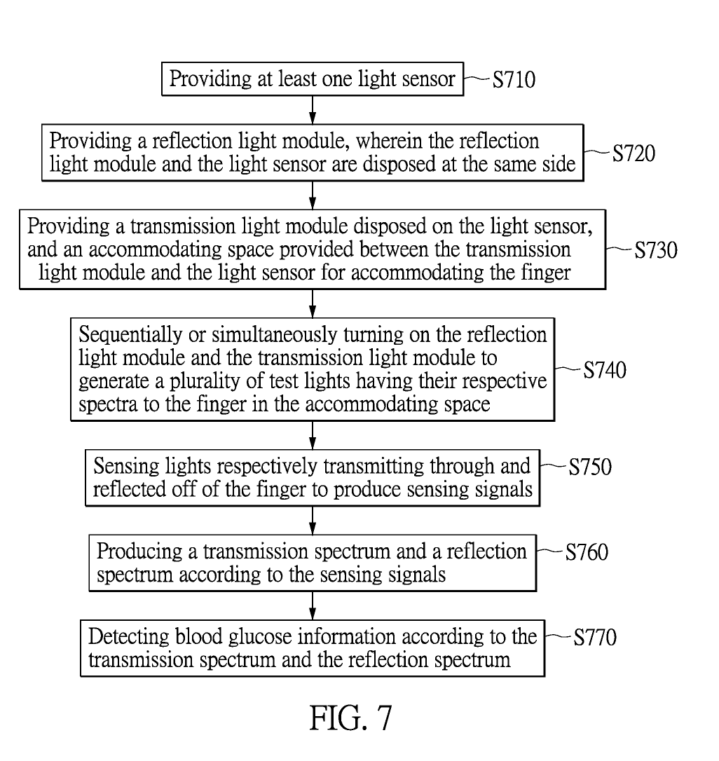

According to the sensing methods shown in FIG. 4-FIG. 6, it can be found that the bio-sensing apparatus 100 can employ different sensing modes to sense the finger 101, and a biometric signals sensing method is therefore provided. Please refer to FIG. 7 which is a flow chart of an embodiment of the biometric signals sensing method of the present disclosure. The steps are as follows: in S710: providing at least one of the light sensors 121, 122; in S720: providing the reflection light module 130, wherein the reflection light module 130 and the light sensors 121, 122 are disposed at the same side; in S730: providing the transmission light module 140 disposed on the light sensors 121, 122, and the accommodating space 160 provided between the transmission light module 14 and the light sensors 121, 122 for accommodating the finger 101; in S740: sequentially or simultaneously turning on the reflection light module 130 and the transmission light module 140 to produce a plurality of test lights having their respective spectra to the finger 101 in the accommodating space 160; in S750: sensing lights respectively transmitting through and reflected off of the finger 101 to produce the sensing signals SS1, SS2; in S760: producing a transmission spectrum and a reflection spectrum according to the sensing signals SS1, SS2, and in S770: the bio-sensing apparatus 100 detecting the blood glucose information according to the transmission spectrum and the reflection spectrum.

As mentioned previously, the bio-sensing apparatus 100 of the present disclosure is capable of respectively sensing the absorption spectrum information about the test light transmitting through the finger 101 and the absorption spectrum information about the test light reflected off of the finger 101, thereby measuring blood glucose level more accurately. By outputting light having different spectra, the present disclosure can obtain a larger spectral range, and by using the wave shape and wave peak with respect to the absorption wavelength, absorbance, and absorption spectrum, the present disclosure can determine blood glucose concentration. In addition, the bio-sensing apparatus 100 and sensing method thereof provided by the present disclosure are able to detect and determine other bio-parameters, thereby promoting the measurement precision and operation speed.

In summary, the bio-sensing apparatus 100 and sensing method thereof provided by the present disclosure are able to sequentially or simultaneously provide lights on and under the finger 101 to sense the finger 101 to obtain blood glucose information more precisely. When detecting the blood glucose level, the bio-sensing apparatus 100 can provide the absorption spectrum information about the test light transmitting through the finger 101 and the absorption spectrum information about the test light reflected off of the finger 101, as well as the sensing information related to different wavebands according to the actual requirements, so as to enable the blood glucose level to be calculated and analyzed more correctly.

The above-mentioned descriptions represent merely the exemplary embodiment of the present disclosure, without any intention to limit the scope of the present disclosure thereto. Various equivalent changes, alterations or modifications based on the claims of present disclosure are all consequently viewed as being embraced by the scope of the present disclosure.

What is claimed is:

1. A bio-sensing apparatus adapted to sense blood glucose information by using one finger, comprising:
at least one light sensor sensing a light intensity signal to produce a sensing signal;
a reflection light module disposed at the same side as the light sensor for producing a first test light;
a transmission light module disposed on the light sensor for generating a second test light, and an accommodating space disposed between the light sensor and the transmission light module, and
a control module electrically connected to the light sensor, the reflection light module and the transmission light module, and receiving the sensing signal to detect the blood glucose information.

2. The bio-sensing apparatus according to claim 1, wherein the reflection light module and the transmission light module both emit light toward the accommodating space.

3. The bio-sensing apparatus according to claim 1, wherein the reflection light module comprises at least one first light array having a plurality of light sources having their respective spectra for generating the first test light, and the first test light has a wavelength range ranging from 700 nm-3000 nm.

4. The bio-sensing apparatus according to claim 1, wherein the transmission light module comprises at least one second light array having a plurality of light sources having their respective spectra for generating the second test light, and the second test light has a wavelength range ranging from 700 nm-3000 nm.

5. The bio-sensing apparatus according to claim 1, wherein the reflection light module comprises a plurality of first light sources and a plurality of first filters, the plurality of first filters are respectively disposed on the plurality of first light sources for producing a spectrum having different wavebands; and the transmission light module comprises a plurality of second light sources and a plurality of second filters, the plurality of second filters are respectively disposed on the plurality of second light sources for producing a spectrum having different wavebands.

6. The bio-sensing apparatus according to claim 1, wherein in a first sensing mode, the reflection light module generates light to the finger in the accommodating space during a first time period, the light sensor senses the received light to produce a first sensing signal, and the transmission light module generates light to the finger in the accommodating space during a second time period, the light sensor senses the received light to produce a second sensing signal, and the bio-sensing apparatus detects the blood glucose information according to the first sensing signal and the second sensing signal, wherein the first time period and the second time period do not overlap.

7. The bio-sensing apparatus according to claim 1, wherein in a second sensing mode, the reflection light module and the transmission light module both generate light to the finger in the accommodating space, the light sensor senses the received light to produce a third sensing signal, and the bio-sensing apparatus detects the blood glucose information according to the third sensing signal.

8. The bio-sensing apparatus according to claim 1, wherein when the bio-sensing apparatus is in the process of sensing, the reflection light module and the transmission light module sequentially or simultaneously generate light having a plurality of spectra to the finger in the accommodating space, the light sensor senses the received light to produce the sensing signals, and the bio-sensing apparatus produces a reflection spectrum and a transmission spectrum according to the sensing signals and then detects the blood glucose information according to the reflection spectrum and the transmission spectrum.

9. The bio-sensing apparatus according to claim 8, wherein a calculation method used to calculate the blood glucose information comprises analysis of variance, regression analysis, or curve fitting.

10. The bio-sensing apparatus according to claim 1, wherein the control module comprises:
a light controller electrically connected to the reflection light module and the transmission light module for adjusting the spectrum of the first test light and the second test light;
an analog/digital converter electrically connected to the light sensor for receiving sensing signals outputted by the light sensor, and then transforming the received sensing signals into digital signals, and
a processing unit electrically connected to the analog/digital converter for receiving the digital signals to detect the blood glucose information.

11. The bio-sensing apparatus according to claim 1, wherein light emitted by the reflection light module and the transmission light module comprise near-infrared and short wavelength infrared.

12. The bio-sensing apparatus according to claim 1, wherein the light sensor comprises a semiconductor photosensitive array having a plurality of semiconductor photosensitive units, and each of the semiconductor photosensitive units comprises a first-type doped semiconductor layer and a second-type semiconductor layer.

13. The bio-sensing apparatus according to claim 1, wherein the light sensor is a photoelectric sensor.

14. The bio-sensing apparatus according to claim 1, wherein the light sensor senses a waveband ranging from 700 nm-3000 nm.

15. A biometric signals sensing method adapted to sense blood glucose information by using one finger, comprising:
providing at least one light sensor;
providing a reflection light module, wherein the reflection light module and the light sensor are disposed at the same side;
providing a transmission light module disposed on the light sensor, and an accommodating space provided between the transmission light module and the light sensor for accommodating the finger;
turning on the reflection light module and the transmission light module to generate a plurality of test lights having their respective spectra to the finger in the accommodating space;
sensing lights transmitting through and reflected off of the finger to produce sensing signals;
producing a reflection spectrum and a transmission spectrum according to the sensing signal, and
detecting the blood glucose information according to the reflection spectrum and the transmission spectrum.

16. The biometric signals sensing method according to claim 15, wherein the plurality of test lights comprise a first test light provided under the finger and a second test light provided on the finger.

17. The biometric signals sensing method according to claim 15, wherein the sensing signals comprise a first sensing signal sensing light reflected off of the finger and a second sensing signal sensing light transmitting through the finger.
